(19) United States
(12) Patent Application Publication
(10) Pub. No.: US 2008/0242739 A1
(43) Pub. Date: Oct. 2, 2008

(54) Mixed hydrophobe polysaccharide as polymeric emulsifier and stabilizer

(76) Inventors: Gijsbert Kroon, Giessendam (NL); Catharina Maria Gortz, Delft (NL); Kate M. Lusvardi, Chadds Ford, PA (US)

Correspondence Address: HERCULES INCORPORATED;Hercules Plaza, 1313 N. Market Street, Wilmington, DE, 19894-0001, US

(21) Appl. No.: 12/079,645

(22) Filed: Mar. 28, 2008

Related U.S. Application Data
(60) Provisional application No. 60/921,092, filed on Mar. 30, 2007.

(51) Int. Cl. A61K 47/38; A61K 47/36; A61Q 99/00

(52) U.S. Cl. 514/781; 514/777

(57) ABSTRACT

Personal care compositions an emulsion has an oil phase, a water phase, and a mixed hydrophobe, non-ionic, water-soluble, hydrophobically modified polysaccharide composition comprising a non-ionic water-soluble polysaccharide backbone having at least one C₃-C₈ short chain hydrophobic group and at least one C₉-C₂₄ long chain hydrophobic group attached thereon. This emulsion can be used in a variety of end use applications including textiles, leather, metal treatments, food, pharmaceuticals, paints, agricultural chemicals, polymerization, cleaning and polishing applications, and ore and petroleum recovery. In particular the emulsion is of use in personal care formulation where the emulsion is a component of a vehicle system of the formulation. At least one active personal care ingredient or electrolytes is also present.

### RELATED APPLICATIONS

This application claims the benefit of U.S. Provisional Application Ser. No. 60/921,092, filed on Mar. 30, 2007, the contents of which are incorporated herein by reference in its entirety.

### FIELD OF INVENTION

The present invention relates mixed hydrophobe polysaccharides, and also relates to a stable fluid emulsion and to its use in personal care products. More specifically, the present invention relates to a mixed hydrophobe polysaccharide used in producing stable aqueous emulsions even in the presence of electrolytes and to its use in a variety of fields such as textiles, leather, and metal treatments, food, cosmetics, pharmaceuticals, and paints, in agricultural chemicals, polymerization, cleaning and polishing, and ore and petroleum recovery. The present invention also relates to cosmetics and dermatological and personal care products, containing the mixed hydrophobe polysaccharides, as aqueous surfactant based formulations.

### BACKGROUND OF THE INVENTION

An emulsion is a mixture of two or more immiscible liquids, one being present in the other in the form of droplets. In the classic emulsion, the oil may either be dispersed in the water (oil-in-water, or o/w, emulsion) or the water dispersed in the oil (water-in-oil, w/o, or inverse emulsion). This terminology is important because the emulsion characteristically assumes the properties of the external, or continuous, phase, a key factor in emulsion formulation and design. For example, an oil-in-water emulsion can be diluted with water or dried by evaporation leaving the other ingredients as a film. The water-in-oil emulsion, on the other hand, cannot be dried.

Emulsions are used in a variety of fields such as textiles, leather, and metal treatments, food, cosmetics, pharmaceuticals, and paints, in agricultural chemicals, polymerization, cleaning and polishing, and ore and petroleum recovery.

Emulsions are inherently unstable systems and the risk of deteriorating during storage is greater than with a non-emulsified-product. Emulsion technology, though seemingly based on simple interfacial principles, is highly complex, especially when dynamic and static conditions are considered.

The properties for emulsions that are most apparent, and thus are usually most important, are: ease of dilution, viscosity, color, and stability. For a given type of emulsification equipment, these properties depend upon (1) the properties of the continuous phase, (2) the ratio of the external (continuous) to the internal, or (discontinuous) phase, (3) the particle size of the discontinuous phase of the emulsion, (4) the relationship of the continuous phase to the particles (including ionic charges) and (5) the properties of the discontinuous phase. In any given emulsion, the properties depend upon which liquid constitutes the external (continuous) phase, i.e., whether the emulsion is o/w or w/o. The resulting emulsion is controlled by the emulsifier (type and amount), the ratio of ingredients, and the order of addition of ingredients during mixing.

The dispersibility (solubility) of the emulsion is determined by the continuous phase. Thus, if the continuous phase is water-soluble, the emulsion can be diluted with water. Conversely, if the continuous phase is oil-soluble, the emulsion can be diluted with oil.

Emulsions can be thin or thick fluids, pastes, or gels and may exhibit thixotropy or dilatency. Viscosity is influenced by (1) the characteristics of the external phase, including additives, (2) the volume ratio of the two phases, and (3) the particle or droplet size. Note that the type of emulsion is not regarded as a major influence on viscosity despite the common belief that o/w emulsions are thinner than w/o. This is true only so far as the oils frequently used are more viscous than water. The viscosity of an emulsion is essentially the viscosity of the external phase as long as it represents more than half of the emulsion's total volume. As the proportion of the internal phase increases, the viscosity of the emulsion increases to the point where the emulsion is no longer fluid. When the volume of the internal phase exceeds the volume of the external phase, the emulsion particles become crowded and the apparent viscosity is partially structural viscosity.

Adding thickeners or gelling agents that are compatible with the emulsifier may increase the viscosity of the continuous phase. Many thickeners, such as carboxymethylcellulose (CMC), methylcellulose (MC), and natural gum or clays may often be added with little or no change in the basic emulsifier. If the thickener or gelling agent is a surfactant in its own right, the overall balance of the emulsifier probably requires readjustment. Emulsion viscosity can often be reduced by increasing the proportion of the continuous phase, usually water. Addition of polar solvents, such as alcohol or acetone that may reduce viscosity, usually cause a marked reduction in emulsion stability. Presumably the emulsifier, being more soluble in the polar solvent, is extracted from the interface which is then weakened. Thickening or thinning of the discontinuous phase usually has little or no effect upon the overall viscosity of the emulsion. In normally fluid o/w polymer emulsions, viscosity differences can be obtained by varying the nature of the adsorbed water structure around each particle by means of a change in surfactant or electrolyte concentration.

Prior to the present invention, there was a need in the personal care industry for a product that could emulsify cosmetic oil-in-water lotions or water-in-oil creams without requiring additional heat and that could stabilize an emulsion by preventing phase separation and creaming.

An emulsion is stable as long as the particles of the internal phase do not coalesce. The stability of an emulsion depends upon: (1) the particle size; (2) the difference in density of the two phases; (3) the viscosity of the continuous phase and of the completed emulsion; (4) the charges on the particles; (5) the nature, effectiveness, and amount of the emulsifier used; and (6) conditions of storage, including temperature variation, agitation and vibration, and dilution or evaporation during storage or use. The stability of an emulsion is affected by almost all factors involved in its formulation and preparation. In formulas containing sizable amounts of emulsifier, stability is predominantly a function of the type and concentration of emulsifier.

Emulsifiers can be classified as ionic or nonionic according to their behavior. An ionic emulsifier is composed of an organic lipophilic group (L) and a hydrophilic group (H). The hydrophilic-lipophilic balance (HLB) is often used to characterize emulsifiers and related surfactant materials. In order to determine a material's HLB, the fraction of material's molecular mass associated with the hydrophilic portion of the material's molecular mass is multiplied by the number 20. This HLB is represented by the equation HLB=(20)(Mh/M) where Mh is the molecular mass of the hydrophilic portion of the molecule, and M is the molecular mass of the whole molecule, giving a result on an arbitrary scale of 0 to 20. An HLB value of 0 corresponds to a completely hydrophobic molecule while a value of 20 would correspond to a completely of hydrophilic molecule.

The ionic emulsifier may be further divided into anionic and cationic emulsifiers, depending upon the nature of the ion-active group. The lipophilic portion of the molecule is usually considered to be the surface-active portion.

Nonionic emulsifiers are covalent in nature and show no apparent tendency to ionize. They can, therefore, be combined with other nonionic surface-active agents and with either anionic or cationic agents as well. The nonionic emulsifiers are likewise less susceptible to the action of electrolytes than are anionic surface-active agents. Solubility of an emulsifier is of great importance in the preparation of emulsifiable concentrates.

Oil-in-water emulsifying agents produce emulsions in which the continuous phase is hydrophilic; hence, such emulsions are generally dispersible in water and will conduct electricity. The surfactants that are capable of producing such emulsions usually have an HLB of more than 6.0 (preferably 7), the hydrophilic portion of their molecules being predominant. (Between HLB 5 and 7 many surfactants will function as either w/o or o/w emulsifiers, depending on how they are used.)

Two important parameters of the emulsion are droplet size and long-term shelf stability over a range of temperatures. Small droplets (i.e., less than 5 microns in diameter) are desired so that emulsions have a high degree of opacity and are easier to stabilize. Long-term shelf stability correlates with rheological parameters such as yield stress and elasticity. Ultimately, formulated lotions must have an acceptable feel to consumers.

A common approach to provide emulsification and stabilization in cosmetic oil-in-water lotions is through a three-dimensional surfactant network which forms upon heating waxy surfactant solutions to greater than 65° C. The network is an association of a large excess of surfactant molecules (i.e., 5-10 wt. %) and shows limited temperature stability (less than 40-45° C.). To improve the stabilizing attributes of the surfactant network, physical gel formers such as Carbopol® crosslinked polyacrylates are typically added to lotions. It is through this combined approach (excess surfactant and physical gel) that both emulsification and long term shelf stability of lotions are achieved. The drawbacks to these cosmetic lotions are the required heat for emulsification and high concentration of surfactant, which can cause skin irritation. A material that could provide both room temperature emulsifying properties and stabilizing properties at low use level is desirable.

Polymeric emulsifiers are hydrophilic polymers that are hydrophobically modified by introducing an alkylic chain. Their chemical structure allows them to act as oil-in-water emulsifiers (and as stabilizers). Hydrophobically modified cross-linked polyacrylic acid copolymers (e.g. Carbopol® ETD 2020 polymers, available from Noveon, Inc.) are used as primary emulsifiers in the cosmetics industry. However, due to their anionic character, they can not be formulated in electrolyte containing emulsions because electrolytes “break” the emulsion and liquefy it.

Another approach for overcoming the instability problem of the oil-in-water emulsions is by strongly increasing the content of emulsifier in these emulsions. However, it is known that emulsifiers, when used in large quantities, can have irritating effects on certain skin types. Moreover, creams obtained from oil-in-water emulsions where high emulsifier content are used are often compact and heavy.

Stabilization of emulsions against flocculation and/or coalescence requires the presence of an energy barrier between the droplets to prevent close approach (whereby the van der Waals attraction is strong). Two general mechanisms may be applied to create such a high (repulsive) energy barrier.

Electrostatic Stabilization
It is based on the formation of an electrical double layer. When two droplets approach to a distance of separation where the double layers begin to overlap, strong repulsion occurs provided the surface or zeta potential is sufficiently high and the electrolyte concentration and valency of the ions is low.

Steric Stabilization
It is based on the adsorption of a surfactant or polymers at the oil/water interface with the hydrophobic (alkyl) group pointing to (or dissolved in) the oil phase and the hydrophilic chain remaining in the aqueous phase. When two droplets approach each other to a separation distance such that the adsorbed layers begin to overlap, repulsion occurs as a result of two mechanisms:
   Unfavorable mixing of the polymer layers when these are in good solvent conditions (osmotic effects); and
   Reduction in configurational entropy on significant overlap (entropic effects)

U.S. Pat. No. 4,904,772 discloses the use of water-soluble, cellulose ether that has at least two hydrophobic radicals having 6 to 20 carbon atoms wherein one of the hydrophobic radicals has a carbon chain length that is at least two carbon atoms longer than that of the other hydrophobic radical. This patent discloses that this cellulose ether can be used in paints, as stabilizers in emulsion polymerization, as protective colloids in suspension polymerization, as thickeners in cosmetics and shampoos, and as flocculent in mineral processing.

U.S. Pat. No. 6,166,078 discloses the use of cetyl modified hydroxyethylcellulose (Polysurf® 67 cetyl hydroxyethylcellulose, available from Hercules Incorporated) in stable gel compositions containing dispersed oil and large quantities of electrolytes. These electrolyte containing emulsions have limited short term shelf stability at elevated temperatures as well as limited long term shelf stability of emulsions without the presence of electrolytes at elevated temperatures.

Hence, a need exists in many industries for an oil-in-water or water-in-oil emulsion that is easy to make without requiring heat and that is stable for long periods of time without phase separation and/or creaming.

### SUMMARY OF THE INVENTION

The present invention relates to a mixed hydrophobe, non-ionic, water-soluble polysaccharide composition with a water-soluble polysaccharide backbone having at least one C₃-C₅ short chain hydrophobic group and at least one C₉-C₂₄ long chain hydrophobic group thereon.

The present invention also relates to an emulsion having an oil phase, a water phase, and a mixed hydrophobe, water-soluble polysaccharide composition with a non-ionic water-soluble polysaccharide backbone having at least one C₃-C₈ short chain hydrophobic group and at least one C₉-C₂₄ long chain hydrophobic group thereon, water-soluble polysaccharide.

The present invention also relates to the use of the emulsion having an oil phase, a water phase, and a mixed hydrophobe, water-soluble polysaccharide composition with a non-ionic water-soluble polysaccharide backbone having at least one C₃-C₈ short chain hydrophobic group and at least one C₉-C₂₄ long chain hydrophobic group thereon, water-soluble polysaccharide in a variety of fields selected from the group consisting of textiles, leather, metal treatments, food, cosmetics, pharmaceuticals, paints, agricultural chemicals, polymerization, cleaning and polishing applications, and ore and petroleum recovery.

This invention is also directed to a skin care composition including the above mentioned emulsion and containing electrolytes and/or at least one active personal care ingredient.

### DETAILED DESCRIPTION OF THE INVENTION

It was surprisingly discovered that a mixed hydrophobe polysaccharide could function as both an emulsifier and stabilizer emulsions. This mixed hydrophobe polysaccharide is of particular use for producing emulsions for use in skin care products. This mixed hydrophobe polysaccharide functionally replaces surfactants in cosmetic emulsions, creams, and lotions at significantly lower use levels.

### Emulsions

In accordance with this invention, emulsions with this mixed hydrophobe polysaccharide have small droplets (i.e., diameters less than 5 micrometers) and rheological properties that surprisingly do not vary with temperature. Thus, emulsion stability is maintained up to 50° C. Small droplets are desired in order for emulsions containing these small droplets have a high degree of opacity and are easier to stabilize. Long term shelf stability of emulsions correlates with rheological parameters such as yield stress and elasticity. This attribute of temperature stability is not typical for structured surfactant or polymer solutions, which “melt” with increasing temperature and cause a loss in emulsion stability at elevated temperatures. Hence, the personal care products of this invention find use in a variety of applications where structure is required over a wide range of temperatures. Notwithstanding, ultimately, formulated lotions must have an acceptable feel to consumers.

In accordance with this invention, emulsions may be oil-in-water or water-in-oil, designating the continuous and discontinuous or internal phases. In general, o/w emulsions conduct electricity, are dilutable with water, feel more like water, dry (lose water) rapidly, can be washed away (off the skin, etc), are more corrosive, and exhibit the aqueous properties of the continuous phase. On the other hand, w/o emulsions conduct electricity poorly if at all, may be diluted with oil or solvents, feel more like oil, resist drying or loss of water, although they lose a volatile solvent readily, are difficult to wash away, are less corrosive, and in general depending upon the oil phase, exhibit the properties of the continuous oil phase.

The particle size of a liquid emulsion is related to the method preparation, the energy input, the viscosity difference between the phases, and the type and amount of surfactant used. With reference to small particle size formation, emulsions may be classified into low emulsifier formulas that require only moderate mechanical effort. Energy input is an important variable. Particle size generally decreases with vigorous agitation, small viscosity difference between the two phases, and the use of a larger amount of the proper surfactant. In this invention, no surfactant is used or needed because the mixed hydrophobe polysaccharide functions as both an emulsifier and stabilizer. Hence, fewer components are needed to form the emulsion with the result that less energy is needed to form an emulsion with small particle size.

In an emulsion, the larger the particle sizes, the greater is the tendency of the particles to coalescence and further increase the particle sizes. Thus, fine particles promote stable emulsions. In this invention, coalescence is retarded by the use of this mixed hydrophobe polysaccharide which provides a protective colloid action.

### Polysaccharides

In accordance with this invention, the polysaccharide polymer for the backbone of the hydrophobically modified polymer is cellulose ether. Examples of cellulose ethers are hydroxyethylcellulose (HEC), hydroxypropylcellulose (HPC), methylcellulose (MC), hydroxypropylmethylcellulose (HPMC), ethylhydroxyethylcellulose (EHEC), and methylhydroxyethylcellulose (MHEC).

Cellulose ethers are widely used as thickeners in personal care products. The size and amount of a hydrophobe used to modify cellulose ether backbone primarily dictate the water-solubility and rheological properties of these hydrophobically modified polymers. For instance, a hydroxyethylcellulose derivative having a long alkyl chain hydrophobe (i.e., chain length of 12 or more carbon atoms) exhibits very high aqueous viscosity at a much lower alkyl content than its shorter alkyl chain (i.e., less than 8 carbons atoms) containing counterparts. However, similar polymers having long alkyl chains become water-insoluble at a lower level of alkyl substitution. This insolubility severely restricts their usefulness in situations where a higher hydrophobe level is best suited to achieve the desired performance properties.

According to the present invention, the polysaccharide polymers have associative, hydrophilic, and hydrophobic properties. The term “associative” when applied to thickeners mean a water-soluble polymer containing hydrophobic groups whose attraction to one another in the aqueous phase and to particles in the dispersed phase both thicken and control the rheology of the emulsion. The term “hydrophilic” means water-loving or attracted to water. The term “hydrophobic” means water-hating or repelled by water. Hence, the different properties in the molecule of the instant invention produce a complex environment that requires a balancing of the components of the molecule for the optimum properties. This balancing lends itself to many possibilities for variations in properties.

In accordance with this invention, the short chain hydrophobic group contains 3 to 8 carbon atoms, preferably the short chain hydrophobic group contains from 3 to 5 carbon atoms, most preferably 4 carbon atoms Examples of such moieties are propyl, butyl, and pentyl radicals. The long chain hydrophobic group containing 9 to 24 carbon atoms. Examples of such moieties are nonyl, hexadecyl, and decyl dodecyl.

Two or more of the following performance properties may be attained simultaneously by controlling the amount present in the molecule of the short chain alkyl hydrophobe, the long chain alkyl hydrophobe, and the hydroxyethyl modification process as well as the molecular weight of the polymers.

SELF-ASSOCIATIVE RHEOLOGY is defined as having the following properties:
a. High yield stress under low shear conditions,
b. Shear thinning viscosity rendering relatively low viscosity under high shear,
c. Reduced elongational viscosity under high speed stretching conditions, and
d. Rapid structure recovery after being subject to transient, high shear.

Moreover, as a result of the self-association of the long chain hydrophobic groups, there will be enhanced yield stress, pseudoplasticity, and thickening efficiency. These properties may lead to an increase in sag resistance in plasters joint compounds, and cement stability of other water-borne dispersions, while allowing desirable workability, extrudability or sprayability in the concerned application. The reduced solution elasticity with lower-DP furnishes can impart to paint spatter resistance and misting resistance in roll/size press applications of coatings and adhesives containing the mixed hydrophobe polysaccharides of the present invention. In applications where spatter or misting is not a primary concern, hydrophobically modified, high-DP HEC polymers also represent a means to attain ultra-high thickening efficiency for reduction of cost-in-use. The mixed hydrophobe polysaccharides of the present invention with a high-DP furnish can yield a substantially higher viscosity than conventional H or HH type of HEC products under low-to-medium shear conditions.

REDUCED MINERAL ADSORPTION can allow:

a. Improved water retention in mineral and mineral/latex containing products and

b. Enhanced thickening efficiency and/or workability in products containing suspended mineral particles

Because of the butylglycidal ether (BGE) substitution, there will be lower adsorption in mineral systems than unmodified HECs for improved water retention and flow properties. While conventional HMHECs may provide the aforementioned rheological properties, their use in mineral-based end products are often limited by the strong interactions between the HEC backbone and key minerals such as clays and cements. The BGE substitution, even at relatively low level of 0.01 or lower, has been found previously to cause a significant reduction in the interaction between HEC and commonly used minerals. The reduced mineral interaction can be attributed to reduced hydrogen bonding as a result of fewer accessible OH-groups on the BGE modified HEC. The solution rheology of the invention has been found to be less temperature sensitive than conventional MHPC over a range of 20 to 45° C. This feature may indicate desirable water retention performance of the invention in relatively hot weather.

CONTROLLED HYDROPHOBICITY/HYDROPHILICITY BALANCE is defined as the control of

a. Surface activity,

b. Thermal flocculation or gelation behavior, and

c. Solvent or monomer miscibility.

This controlled hydrophobicity attribute can lead to low surface or interfacial tension, which can be beneficial to creams, lotion, shampoos, printing and other applications.

TABLE 1
Compositions and properties of representative associative HMHEC-B samples

| | Hopewell designation # | | | | |
|---|---|---|---|---|---|
| | 0815-60 | 0815-56 | 0819-28 | 0819-34 | 0819-32 |
| Furnish | Buckeye HVE | Buckeye HVE | Buckeye HVE | Ethenier FUHV | Columbus Fluff |
| 4% Brookfield viscosity, cP | 5047 | 8213 | 9107 | 9327 | 826 |
| Cloud Point in water, Deg. C. | —* | —* | 60 | 80 | 50 |
| Surface Tension of 1% Solution | 40.7 | 34 | 48.2 | 54.5 | 54.7 |
| HE-MS | 2.27 | 2.95 | 3.12 | 2.93 | 3.08 |
| BGE-DS | 0.087 | 0.108 | 0.067 | 0.062 | 0.072 |
| CGE-DS | 0.007 | 0.007 | 0.008 | 0.007 | 0.008 |
| Mw | 7.43E+05 | 7.82E+05 | 7.51E+05 | 8.23E+05 | 3.79E+05 |

*Cloud points were not measured at Hopewell due to initial sample cloudiness.

Table 2 below lists the above attributes against the needs of some potential applications. As indicated by Table 2, the invention polymers are potentially useful in a variety of applications including, ceramic extrusion, joint compound, plasters, tile cements, masonry cements, high PVC paints, paper coatings, metering size press, skin and hair care products such as creams/lotions and shampoos, adhesives, and fountain inks.

TABLE 2
Attributes of invention versus needs of potential applications

| | Reduced | Yield | Pseudo- | Thick. | Spatter/ | Thermal | Surface |
|---|---|---|---|---|---|---|---|
| | Interactn | Strength | Plasticity | Eff. | Mist | Floc. | Activity |
| Extruded | X | X | X | | | X | |
| Ceramics | | | | | | | |
| Plasters | X | X | X | X | | | |
| Tile Cement | | | | | | | |
| Joint Cpd. | X | X | X | X | | | X |
| Mortar | | | | | | | |
| Cement | | | | | | | |
| High PVC | X | X | X | X | X | | |
| Paints | | | | | | | |
| Cream/Lotion | X | X | X | X | | | X |
| Shampoo | | | | | | | |
| Paper Coating | X | X | X | X | X | | |
| Size Press | | | | | | | |
| Adhesives, | | X | X | X | X | | X |
| Inks & Others | | | | | | | |

According to the present invention, the mixed hydrophobe polysaccharides can be used in construction, ceramic extrusion, paper coating/size press, paint, and personal care. More specifically, the self-associative HMHEC-B material may be used as a rheology modifier/binder in joint compounds, cement or gypsum based plasters, flat paints, extruded ceramics, creams, lotions, shampoos, paper coatings, and other water-borne products.

The mixed hydrophobe polysaccharides of this invention can be prepared directly from cellulose. First a cellulose source, such as chemical cotton, is added to and reacted with a mixture of an inert organic diluent and alkali metal hydroxide to form an alkali cellulose. Then, ethylene oxide or another substituent is added to the resultant alkali cellulose and once the reaction is completed the product is treated with nitric acid. To this reaction mixture is added an alkyl glycidylether and, optionally, a second increment of ethylene oxide. After the reaction is completed, the product is then neutralized, filtered, washed with aqueous inert diluents, and dried.

More specifically, the preferred procedure for preparing a polymer using alkyl bromides in an alkylyzation reaction of cellulose in mixture of t-butyl alcohol, ispropyl alcohol, acetone, water and sodium hydroxide under a nitrogen atmosphere for a period of time that is sufficient to distribute the alkali onto the cellulose. Then, ethylene oxide is added to the alkali cellulose slurry, followed by heating at about 70° C. for about one hour. The resulting slurry is partially neutralized and additional ethylene oxide is added to the reaction mixture. Then, the resulting reaction mixture is heated at about 90-95° C. for about 90 minutes. Caustic and alkyl bromides (two different alkyl bromides, one having 3-8 carbon atoms and the other having 9-24 carbon atoms) are added, followed by heating of the reaction mixture at about 115° C. for about 2 hours and neutralization of the reaction mixture. The reaction mixture is washed and then the resultant polymer is purified.

Another method for preparing the polymer of the present invention is to start from a commercial intermediate product. Briefly, the modifications can be effected by slurrying a polymer, such as hydroxyethylcellulose, in an inert organic diluent such as a lower aliphatic alcohol, ketone, or hydrocarbon and adding a solution of alkali metal hydroxide to the resultant slurry at a low temperature. When the ether is thoroughly wetted and swollen by the alkali, a mixture of alkylglycidyl ethers is added and the reaction is continued with agitation and heating until completed. Residual alkali is then neutralized and the product is recovered, washed with inert diluents, and dried.

In accordance with the present invention, the mixed hydrophobe polymers have a weight average molecular weight (Mw) generally with a lower limit of 50,000 Daltons (Da), preferably 100,000 Da, and more preferably 300,000 Da. The upper limit of the molecular weight is generally 600,000 Da, preferably 700,000 Da and more preferably 1,000,000 Da. The backbone has at least one short chain hydrophobic group composed of C₃-C₈, preferably C₃-C₅, and more preferably C₄ and has at least one long chain hydrophobic group composed of C₉-C₂₄, preferably C₁₄-C₂₂, and more preferably C₁₄-C₁₈. The short chain hydrophobic group content is at least 0.5 wt % of the mixed hydrophobe polymers of the present invention. The long chain hydrophobic group content is at least 0.2 wt % of the mixed hydrophobe polymers of the present invention.

### Personal Care Products

According to the present invention, personal care products are defined as any formulation that is used to protect or treat or clean or enhance the appearance of a human being. The personal care composition normally has 1) a vehicle system which is composed of normally a thickener and solvent, and 2) an active personal care ingredient.

According to the present invention, the solvent used in the vehicle system should be compatible with the other components in the present composition. Examples of the solvents used in the present invention are water, water-lower alkanols mixtures, and polyhydric alcohol having from 3 to 6 carbon atoms and from 2 to 6 hydroxyl groups. Preferred solvents are water, propylene glycol, water-glycerine, sorbitol-water, and water-ethanol. The solvent (when used) in the present invention is present in the composition at a level of from 0.1% to 99% by weight of the composition.

Personal care products are available in different product forms. For example: solutions, colloidal solutions, emulsions and microemulsions (e.g. o/w and w/o), multiple emulsions (e.g. w/o/w), dispersions, solubilizations, pastes, oils, foams, powders, sticks, bars, gels and aerosols.

The active personal care component can be optional in certain compositions because the vehicle system can be the active ingredient component. An example of this is the use of the vehicle system in a denture adhesive as either a cream or powder. However, when an active personal care ingredient is needed, it must provide some benefit to the user's body. Examples of substances that may suitably be included in the personal care products according to the present invention are as follows:

1) Perfumes, which give rise to an olfactory response in the form of a fragrance and deodorant perfumes which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone oils, mineral oils and vegetable oils which give rise to a tactile response in the form of an increase in skin lubricity;

4) Deodorants other than perfumes, whose function is to reduce the level of or eliminate microflora at the skin surface, especially those responsible for the development of body malodor. Precursors of deodorants other than perfumes can also be used;

5) Antiperspirant actives, whose function is to reduce or eliminate the appearance of perspiration at the skin surface, are particularly advantageously selected from the group consisting of aluminum chlorhydrate and aluminum zirconium chlorhydrate;

6) Moisturizing agents, that keep the skin moist by either adding moisture or preventing it from evaporating from the skin, examples of advantageous moisturizing agents are: e.g. glycerin, sorbitol, propylene glycol, polyethyleneglycols with M_{w} 200 to 600 Da, sorbeth-30, lactic acid and/or sodiumlactate, methyl glucoside alkoxylates;

7) Cleansing agents, that remove dirt and oil from the skin, examples of advantageous cleansing agents are: e.g. Na, NH₄ laureth-2 sulfate, Na, NH₄ laureth-3 sulfate, alpha olefin sulfonate, TEA or Na lauryl sulfate, NH₄ lauryl sulfate;

8) Sunscreen active ingredients that protect the skin and hair from UV and other harmful light rays from the sun. In accordance with this invention, a therapeutically effective amount will normally be from 0.01 to 10% by weight, preferably 0.1 to 5% by weight, of the personal care products.

The personal care products according to the invention may comprise at least one UV-A filter substance and/or at least one UV-B filter substance and/or at least one further (soluble or insoluble) inorganic pigment selected from the group consisting of the oxides of iron, zirconium, silicon, manganese, aluminum, cerium and mixtures thereof and also modifications in which the oxides are the active ingredients.

If the emulsions according to the invention contain UV-B filter substance, the latter may be oil-soluble or water soluble. Examples of oil-soluble UV-B filters which are advantageous according to the invention are:
3-benzylidenecamphor derivatives, preferably 3-(4-methylbenzylidne)camphor, 3-benzylidenecamphor;
4-aminobenzoic acid derivatives, preferably 2-ethylhexyl-4-(dimethylamino)-benzoate, amyl 4-(dimethylamino)benzoate;
Esters of cinnamic acid, preferably 2-ethylhexyl 4-methoxycinnamate, isopentyl 4-methoxycinnamate;
Esters of salicylic acid, preferably 2-ethylhexyl salicylate, 4-isopropylbenzyl salicylate, homomethyl salicylate;
Derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;
Esters of benzalmalonic acid, preferably di(2-ethylhexyl)-4-methoxybenzalmalonate;
Benzotriazole derivatives, preferably 2,2′-methylenebis(6-(2H-benzotriazol-2-yl)-4-(1,1,3,3-tetramethylbutyl)phenol)

Examples of advantageous water-soluble UV-B filter substances are
salts of 2-phenylbenzimidazole-5-sulphonic acid, such as its sodium, potassium or its triethanolammonium salt, and also the sulphonic acid itself;
sulphonic acid derivatives of 3-benzylidenecamphor, such as e.g. 4-(2-oxo-3-bornylidenemethyl)benzenesulphonic acid, 2-methyl-5-(2-oxo-3-bornylidene-methyl)sulphonic acid and their salts.

A list of said UV-B filters, which may be used in the emulsions according to the invention, which is of course not intended to be limiting, is as follows: PABA=p-aminobenzoic acid, camphor benzalkonium methosulfate, phenylbenzimidazole sulfonic acid, terephthalyidene dicamphor sulfonic acid, benzylidene camphor sulfonic acid, benzophenone-4 (acid) and benzophenone-5 (sodium salt).
It can also be advantageous to use, in the emulsions according to the invention, UV-A filters which have been customarily present in cosmetic products.
These substance are preferably derivatives of dibenzoylmethane, in particular 1-(4′-tert-butylphenyl)-3-(4′-methoxyphenyl)proane-1,3-dione and 1-phenyl-3-(4′-isopropylphenyl)propane-1,3-dione.
Further advantageous UV-A filter substances are phenylene-1,4-bis(2-benzimidazyl)-3,3′-5,5′-tetrasulphonic acid and its salts.
Advantageous UV filter substances are also so-called broad-band filters, i.e. filter substances which absorb both UV-A and UV-B radiation. A broad-band filter which is to be used advantageously is, for example, ethylhexyl 2-cyano-3,3-diphenylacrylate (octocrylene).

9) Hair treatment agents, that conditions the hair, cleans the hair, detangle hair, act as styling agent, anti-dandruff agent, hair growth promoters, hair dyes and pigments, hair perfumes, hair relaxer hair bleaching agent, hair moisturizer, hair oil treatment agent, and antifrizzing agent;

10) Oral care agents, such as dentifrices and mouth washes, that clean, whiten, deodorize and protect the teeth and gum;

11) Denture adhesives that provide adhesion properties to dentures;

12) Shaving products, such as creams, gels, and lotions and razor blade lubricating strips;

13) Tissue paper products, such as cleansing tissues; and

14) Beauty aids, such as foundation powders, lipsticks, and eye care.

The above list is only examples and is not a complete list of active ingredients that can be used in personal care compositions. Other ingredients that are used in these types of products are well known in the personal care industry.

In addition to the above ingredients conventionally used in products for personal care, the composition according to the present invention can optionally also include ingredients such as colorants, preservatives, antioxidants, vitamins, activity enhancers, spermicidals, emulsifiers and fats and oils.

The vehicle systems in personal care compositions of the present invention can be made using conventional formulation and mixing techniques. Methods of making various types of personal care compositions are described more specifically in the following examples.

The following examples are merely set forth for illustrative purposes, but it is to be understood that other modifications of the present invention can be made without departing from the spirit and scope of the invention. All percentages and parts are by weight, unless specifically stated otherwise.

### EXAMPLE 1

Several commercial and developmental hydrophobically modified (HM) polymers were evaluated as a polymeric emulsifier/stabilizer in 10 wt % neutral oil (Miglyol® neutral oil, available from Condea Chemie GmbH) oil-in-water emulsions at 0.9-wt % polymer. The polymers tested included a range of C₁₆ modified, CM (carboxyl methyl) C₁₆ modified, C₄/C₁₆ modified, C₁₂ modified, C₄ modified, and unmodified hydroxyethylcellulose (HEC). Emulsion droplet size, stability, and rheology were characterized.

All C₁₆ modified cellulose derivatives were excellent at emulsifying and reducing the droplet size to ˜5 microns. The lowest Mw polymer with the highest hydrophobe modification was the most efficient. Higher Mw polymers were only slightly less effective at droplet size reduction. Adding a carboy methyl group had little effect on emulsifying capabilities. On the other hand, polymers modified with alkyl chains containing less than sixteen carbons (e.g., C₁₂, C₄, and no modification), showed a marked decrease in their ability to lower interfacial tension and reduce droplet size. At lower concentrations, emulsion droplet size grows and droplet polydispersity increases for all HMHEC's investigated.

To prevent creaming in a system with droplets on the order of 5 microns, the emulsions must display a yield stress to balance gravitational effects. The relationship of the yield stress to droplet size is an important determination of emulsion stability—the larger the droplets, the larger the yield stress required to prevent droplet migration to the container top. For typical cosmetic oils, the minimum yield stress required to stabilize droplets 1-20 microns in size ranges from 2-40 Pa².

Table 3 shows Theological parameters of the emulsions that remained stable for 2 weeks at 50° C. Only the emulsions formulated with high molecular weight HM polymers showed high elasticity (G′) and a yield stress; a result of both entanglement and hydrophobe interactions. Emulsions stabilized with developmental C₄/C₁₆ cellulosic polymers (0819-26 and 0819-34) exhibited the highest elasticity and yield stress, perhaps due to heterogeneous C₄/C₁₆ substitution resulting in stronger associations. For the emulsions which showed no yield or elasticity, instability was driven primarily by Stokes Law; faster creaming with lower viscosity polymer solutions like Plus 330 and AQU D3441, with less dense oils such as light mineral oil (Drakeol® 7 LT mineral oil, available from Penreco), and with emulsions having large droplets.

TABLE 3
Rheological Parameters of 10-wt % Miglyol Oil-in-Water Emulsions

| | Complex | | | Yield | | |
|---|---|---|---|---|---|---|
| 0.9 wt % | Viscosity | G′ | G″ | Stress | Stress @ | Temp. Dependence/ |
| Polymer | (cP)* | (Pa)* | (Pa)* | (Pa) | G′ = G″ | G′, G″, cross-over T |
| C4/C16 | 4,670 | 27 | 13 | 22 | 70 | Linear decrease @ |
| (0819-26) | | | | | | T > 25° C./no |
| C4/C16 | 4,530 | 26 | 12 | 25 | 70 | Constant/no |
| (0819-34) | | | | | | |
| HM Cotton | 1,440 | 7 | 6 | 10 | 19 | Linear decrease @ |
| Linter | | | | | | T > 25° C./38° C. |
| (3360) | | | | | | |
| Polysurf ®-67 | 2,760 | 16 | 8 | 17 | 40 | Linear decrease @ |
| cetyl HEC | | | | | | T > 25° C./55° C. |
| Plus 430 | 1,640 | 9 | 5 | 19 | 30 | Linear decrease @ |
| | | | | | | T > 25° C./50° C. |

*As measured in linear visco-elastic region.

Additionally, long-term shelf stability over a range of temperature conditions is an important parameter of cosmetic emulsions. Rheologically, this translates to emulsions that do not lose viscosity or elasticity as a function of temperature. As the temperature increases, emulsions stabilized with HM polymer solutions typically show a loss in structure, reaching a point where the elastic component (G′) no longer dominates the viscous component (G″), the overall viscosity of the solution decreases, and creaming occurs rapidly. This happens when thermal energetics begin to dominate the hydrophobic associative energetics. As shown in Table 3, C₁₆ modified polymers typically show this linear decrease in viscoelastic properties with increasing temperatures, and the elastic component (G′) crosses the viscous component (G″) at temperatures less than 50° C. Surprisingly, the emulsion formulated with a C₄/C₁₆ mixed hydrophobe polysaccharide (0819-34) exhibited temperature insensitive rheological parameters, with no decrease in viscosity or loss in elasticity as the temperature was increased. Table 3A, infra, shows that these C₄/C₁₆ mixed hydrophobe polysaccharide have 60-80° C. cloud points in water. It is likely that as the temperature is raised, the usual loss in hydrophobic associations may be balanced by new associations that form as the polymer undergoes conformational changes in reaching its cloud point. This translates to improved emulsion stability at elevated temperatures; an important attribute of cosmetic oil-in-water emulsions.

The C₄/C₁₆ mixed hydrophobe polysaccharide of this invention shows significantly improved emulsion stabilization properties. Emulsions made with this polymer have small droplets and rheological properties that surprisingly do not vary with temperature.

TABLE 3A
Compositions and properties of associative HMHEC-B samples.

| | Designation # | |
|---|---|---|
| | 0819-26 | 0819-34 |
| Furnish | Buckley HVE | Ethenier FUHV |
| 1% Brookfield viscosity, cP | 9,107 | 9,327 |
| Cloud point in water, EC | 60 | 80 |
| Surface Tension of 1.0% solution | 48.2 | 54.5 |
| HE-MS | 3.12 | 2.93 |
| BGE-DS | 0.067 | 0.062 |
| CGE-DS | 0.008 | 0.007 |
| Mw (Da) | 7.51E+05 | 8.23E+05 |

### EXAMPLE 2

To further investigate the mixed hydrophobe polysaccharide C₄/C₁₆ mixed hydrophobe modified cellulose ether, a commercial lotion formulation (Table 4) containing the C₄/C₁₆ mixed hydrophobe polysaccharide (0819-34) as the only emulsifier was prepared at two concentrations, 0.7 wt % and 0.9 wt %.

At elevated temperatures (50° C.), the lotion containing 0.7 wt % polymer began to cream within 6 days. The formulation containing 0.9 wt %, however, has remained stable for over 5 weeks at 50° C. Rheological parameters are shown in Table 5. At 0.7 wt % polymer, the emulsion had a significantly lower yield stress and exhibited a slight decrease in viscoelastic properties as the temperature was raised, hence, creaming ensued. At the 0.9 wt % use-level, however, the yield stress was adequate, no change in rheological properties as a function of temperature was apparent, and the emulsion remained stable at elevated temperatures. The critical concentration for achieving this rheology lies between 0.7 and 0.9 wt % polymer.

This formulation demonstrates the positive aspects of using a single polymer such as the mixed hydrophobe polysaccharide for both emulsification and stabilization: no heat was required during emulsification, no surfactants or co-surfactants were required for achieving stability at elevated temperatures, no neutralization was required to trigger thickening, and a blend of different emollients could be emulsified to tailor the lotion feel.

TABLE 4
Moisturizing lotion which delivers a high level of protection.

| Phase | Ingredient | Wt % | Function |
|---|---|---|---|
| Oil | Avocado Oil | 4.00 | Emollient |
| | Isostearyl Isostearate | 4.00 | Emollient |
| | Octyl Stearate | 3.00 | Emollient |
| | Isosiopropyl Myristate | 3.00 | Emollient |
| | Propylene Glycol | 4.00 | Emollient |
| | Sostearate | | |
| Water | Glycerine | 2.00 | Humectant |
| | Germaben II | 0.50 | Preservative |
| | Associative HMHEC-B | 0.7 or 0.9 | Polymeric |
| | | | Emulsifier |
| | Water | q.s. | Vehicle |

TABLE 5
Rheological Parameters of Moisturizing Lotion

| C₄/C₁₆ | | | | | | Temp. |
|---|---|---|---|---|---|---|
| concen- | Complex | | | Yield | | Dependence/ |
| tration | Viscosity | G′ | G″ | Stress | Stress@ | G′, G″ |
| (wt %) | (cP)* | (Pa)* | (Pa)* | (Pa) | G′ = G″ | cross-over T |
| 0.7 | 1,710 | 10 | 4 | 5 | 9 | Linear decrease @ |
| | | | | | | T > 25° C./none |
| 0.9 | 7,460 | 45 | 14 | 20 | 55 | Constant/none |

A commercial lotion formulation containing a range of emollients and less than 1.0 wt % of this polymeric emulsifier have remained stable at 50° C. exhibiting the dual functionality of this material. Ultimately, formulated lotions must have an acceptable feel to consumers.

Rheological data was collected on a Bohlin CS Rheometer. Dynamic mechanical properties were measured including the storage and loss modulus, complex viscosity, and yield stress. The test conditions are shown below:

| | Temperature | | |
|---|---|---|---|
| | Sweep | Stress Sweep | Yield Stress Test |
| Measuring System | PP 40 | PP 40 | CP 4/40 |
| | (25° C.-65° C.) | | |
| Stress | Automatic | | 6.0E−02-1.0E+02 |
| Frequency | 1 Hz | 1 Hz | N/A |
| Temperature | 5° C./60 seconds | 25° C. | 25° C. |
| Measurement | 20 | 5 | 5 |
| Interval (seconds) | | | |
| Gap | 1 mm | 1 mm | N/A |

### EXAMPLE 3

This Example shows an emulsion containing the C₄/C₁₆ mixed hydrophobe polysaccharide used in an inorganic formulation sunscreen lotion. This formulation was prepared to demonstrate the stability of the emulsion containing the C₄/C₁₆ mixed hydrophobe polysaccharide in inorganic systems that have different environments. In this case, the formulation was stable at 50° C. for more than a week.

TiO₂ Based Sunscreen Lotion

| | | | Formulation Wt |
|---|---|---|---|
| Phase | Chemical Name | Trade Name | % |
| A | Deionized H₂O | — | 67.1 |
| | C₄/C₁₆ x 32071-19-6 | — | 0.7 |
| | Propylene Glycol | Prisorine 2034 | 5 |
| | Disodium EDTA | — | 0.1 |
| B | AllylC₁₂₋₁₅ Alkyl Benzoate | Finsolv TN | 3 |
| | Butyl Stearate | Kessco BS COS | 3 |
| | Myristyl Myristate | Schercemol MM | 4 |
| | Sorbistan Oleate | Span 80 | 0.1 |
| D | Titanium Dioxide | MT100SA | 6 |
| | Octyl Palmitate | Lexol EHP | 9 |
| | Polyglyceryl-10 Decaoleate | Drewpol 10-10-0 | 1 |
| C | Germaben II | — | 1 |

### Procedure

1. Added materials of Phase A to a 70° C. jacketed flask and stirred at setting 4 on a Braun high speed mixer.
2. Mixed Phase B together in a separate jacketed flask and heated to 70° C. until the materials melted.
3. Added Phase B to Phase A and stirred for 2 minutes.
4. Added Phase C to the mixture of A/B and stirred for 2 minutes
5. Mixed Phase D together in a separate flask using a spatula.
6. Mixed Phase D to the mixture of A/B/C and stirred for 2 minutes on the Braun mixer.
7. Continued mixing the mixture of A/B/C/D for an additional 10 minutes.
8. Then, cooled the mixture A/B/C/D to 25° C. while continued stirring on the Braun mixer.
9. Adjusted the cooled mixture A/B/C/D to a pH 7 and then mixed with Braun high speed mixer for 4 minutes.

### Properties

pH . . . 7.12
Appearance . . . white and creamy, glossy
Stability . . . >1 wk at 50° C.

### EXAMPLE 4

This Example shows an emulsion containing the C₄/C₁₆ mixed hydrophobe polysaccharide for use in an organic formulation sunscreen lotion. This formulation was prepared to demonstrate the stability of the emulsion containing the C₄/C₁₆ mixed hydrophobe polysaccharide in organic systems that have different environments. In this case, the formulations were stable at 50° C. for more than a week.

High SPF Organic Sunscreen Cream

| | | | Formulation Wt |
|---|---|---|---|
| Phase | Chemical Name | Trade Mark | % |
| A | Deionized Water | — | 63.6 |
| | C₄/C₁₆ X32071-19.6 | — | 0.7 |
| B | Cetyl alcohol | Crodacol C-70 | 3.3 |
| | Stearyl alcohol | Crodacol S-70 | 3.3 |
| C | Benzophenone 3 | — | 5.1 |
| | Octyl Methoxycinnamate | Neo-Heliopan AV | 7.6 |
| | Octyl Salicylate | Escalol 587 | 5.1 |
| | Mentyl Antranilate | Neo-Heliopan MA | 5.1 |
| D | Octyl Stearate | Estol 1545 | 5.1 |
| E | BHT | — | 0.1 |
| | Germaben II | — | 1.0 |

### Procedure

1. Added materials in Phase A to a 70° C. jacketed flask and stirred at setting 4 on a Braun mixer.
2. Mixed Phase B together on a Braun mixer in a separate jacketed flask and heated to 70° C. until the materials melted.
3. Added Phase B to Phase A and stirred for 2 minutes on a Braun mixer to form mixture A/B.
4. Added Phase C to the mixture A/B and stirred for 2 minutes on the Braun mixer to form mixture A/B/C.
5. Added Phase D to the mixture A/B/C and stirred for 2 minutes to form mixture A/B/C/D.
6. Continued mixing the A/B/C/D mixture for 10 minutes on the Braun mixture.
7. Then, cooled this mixture to 25° C. while stirring. Added Phase E to the mixture A/B/C/D when its temperature was below 45° C. to form mixture A/B/C/D/E.
8. Adjusted the pH of mixture A/B/C/D/E to pH 7.

### Properties

pH . . . 7.01
Appearance . . . white and creamy, glossy
Stability . . . >1 wk at 50° C.

### EXAMPLE 5

Several commercial and developmental hydrophobically modified (HM) hydroxyethylcelluloses were evaluated as polymeric emulsifier/stabilizer in 10 wt % mineral oil-in-water emulsions at 0.5 wt % and 1.0 wt % polymer. See Table 6.

TABLE 6
Properties of HMHEC samples

| | | Properties | |
|---|---|---|---|
| Type | | Hydrophobe(wt %) | MW (Da) |
| HMHEC with low MW, medium | Natrosol ® Plus | C16 = 0.6 | 300K |
| C₁₆ wt % | 330 HEC | | |
| | Natrosol ® Plus | C16 = 0.7 | 300K |
| | 331 HEC | | |
| HMHEC with medium MW, medium | Polysurf ® 67 | C16 = 0.52 | 600K |
| C₁₆ wt % | cetyl HEC | | |
| HMHEC with high MW, C₈C₂₂ | ADPP 4946 | C8 = 0.55 C22 = 0.31 | 1000K |
| modified | ADPP 4947 | C8 = 1.32 C22 = 0.14 | 1000K |
| HMHEC with high MW, C₄C₁₆ | ADPP 4690 | C4 = 3.17 C16 = 0.61 | 1000K |
| modified | ADPP 4627 | C4 = 2.87 C16 = 0.55 | 1000K |

10 wt % oil-in water emulsions were prepared by a) preparing an aqueous stock solution of polymeric emulsifier/stabilizer, b) adding an oil and the preservative to the aqueous polymer solutions to form mixtures, and c) mixing these mixtures in a Braun rotary blender on high speed for 3 minutes to form an emulsion. The composition of the emulsion is given in Table 7.

TABLE 7
Composition oil-in-water emulsion

| Ingredients | Wt % |
|---|---|
| Distilled water | q.s. to 100.0 |
| Polymeric emulsifier/stabilizer | 1.00 |
| Carnation oil (mineral oil) | 10.00 |
| Germaben II (preservative) | 0.20 |

The emulsions formulated with the following polymers did not show creaming or phase separation upon 4 weeks storage at room temperature and 40° C.:

HMHEC with medium molecular weight and medium C₁₆ wt %:
Polysurf® 67 cetyl HEC

HMHEC with high molecular weight and mixed hydrophobes:
C₈ and C₂₂ modified: ADPP 4946
C₄ and C₁₆ modified: ADPP 4690

Acrylates/C₁₀₋₃₀ Alkyl Acrylates crosspolymer: Carbopol ETD 2020

The emulsions formulated with the following polymers did not show creaming or phase separation upon 4 weeks storage at 50° C.:

HMHEC with high molecular weight and mixed hydrophobes:
C₈ and C₂₂ modified: ADPP 4946
C₄ and C₁₆ modified: ADPP 4690

Acrylates/C₁₀₋₃₀ Alkyl Acrylates crosspolymer: Carbopol ETD 2020

A temperature swing test (Controlled Stress Rheometer, Bohlin CS, 1 Hz) was also conducted on these emulsions. The temperature profile was from 25° C. to 60° C. The visco-elastic parameters (G′, G″, Tan δ) were measured as a function of the temperature. The storage modulus G′ described the elastic, gel-like behavior of the sample whereas the loss modulus G″ characterized the viscous, fluid-like behavior.

The emulsions formulated with Carbopol ETD 2020 and mixed hydrophobe HEC showed no crossover point and G′>G″ in the total temperature range. See Table 8.

TABLE 8
Rheological properties of 10 wt % mineral oil-in-water emulsions

| | Rheological properties | | |
|---|---|---|---|
| Polymeric | δ (°) @ | δ (°) @ | δ (° C.) @ |
| emulsifier/stabilizer (wt %) | 25-40° C. | 40-60° C. | G′ = G″ |
| Carbopol ETD 2020 (0.5) | 8.5-6.5 | 6.5-6 | >60 |
| Natrosol ® Plus 330 HEC (1.0) | G′ < G″ | G′ < G″ | — |
| ADPP 4627 (1.0) | 25-30 | 30-30 | >60 |
| ADPP 4690 (1.0) | 25-30 | 30-35 | >60 |
| ADPP 4946 (0.5) | 25-30 | 30-30 | >60 |
| ADPP 4946 (1.0) | 20-25 | 25-28 | >60 |
| ADPP 4947 (0.5) | 30-30 | 30-16 | 38 |
| ADPP 4947 (1.0) | 25-35 | 35-35 | >60 |
| Polysurf ® 67 cetyl HEC (1.0) | 30-35 | 35-35 | 42 |

### EXAMPLE 6

This Example shows the use of C₄C₁₆ HEC in oil-in-water emulsion using large quantities of electrolytes. The composition of the emulsion is given in Table 9.

TABLE 9
Oil-in-water emulsion

| | |
|---|---|
| Ingredients | Wt % |

| | | |
|---|---|---|
| Distilled water | q.s. to 100.0 | q.s. to 100.0 |
| Polymeric emulsifier/stabilizer | 1.00 | 1.00 |
| Calciumchloride | 3.0 | — |
| Calciumnitrate | — | 6.0 |
| Carnation oil (mineral oil) | 10.0 | 10.0 |
| Germaben II (preservative) | 0.20 | 0.20 |

Procedure:
Prepared a stock solution of polymeric emulsifier/stabilizer;
Electrolytes were added to polymer solution
Added mineral oil and Germaben II to the aqueous phase; and
Mixed the formulation with a Braun kitchen mixer for 3 minutes at speed 5 to form the emulsion.

As expected, Carbopol ETD 2020 showed immediately phase separation in all electrolyte containing emulsions. The electrolyte formulation with 1.0 wt % Polysurf® 67 cetyl HEC product showed phase separation after one week at 50° C. Addition of electrolytes resulted in a higher emulsion viscosity of the mixed hydrophobe hydroxyethylcellulose containing emulsion due to the associating side groups.

It was found that 1.0 wt % ADPP 4690 (C₄C₁₆ modified hydroxyethylcellulose) emulsions with 3.0 wt % calcium chloride and 6.0 wt % calcium nitrate were at least stable for 4 weeks at room temperature and 50° C.

### EXAMPLE 7

To further investigate the mixed hydrophobe modified HEC, several mixed hydrophobe modified HECs were evaluated as a polymeric emulsifier/stabilizer in commercial oil-in-water emulsions at 1.0 wt %. The polymers tested included C₈C₂₂ modified HEC and various C₄C₁₆ modified HECs differing in molecular weight and hydrophobe substitution. The composition of the emulsion is given in Table 10.

TABLE 10
Oil-in-water emulsion formulation

| Ingredients | Wt % |
|---|---|
| Water Phase | |
| Distilled water | q.s. to 100.0 |
| Glycerin | 5.0 |
| PEG-40 Stearate | 3.0 |
| Emulsifier/Stabilizer | 1.0 |
| Oil Phase | |
| Carpylic/Capric Triglyceride | 3.3 |
| Octyldodecanol | 3.3 |
| Dicaprylyl Carbonate | 3.3 |
| Phenoxyethanol + Methylparaben + Ethylparaben + | 0.5 |
| Butylparaben + Isobutylparaben + Propylparaben | |

### Procedure:

Polymer (thickener) was added to a mixture of water and glycerine in a flask, while heating (to 50° C.) and mixing (in a blade mixer, @ 700 rpm), until the polymer dissolved completely.
PEG-40 stearate was added.
The oil phase was mixed in a separate flask.
The oil phase was then added to the water phase in the blade mixer (@ 700 rpm), while cooling to ambient temperature.
The mixture was then stirred and homogenized (in a Braun kitchen mixer for 3 minutes at speed 5).

The high molecular weight C₈C₂₂ modified HEC (ADPP 4946) containing emulsions were stable for over 4 weeks at 40° C.

Low to medium molecular weight C₄C₁₆ modified HECs containing emulsions showed immediately phase separation or upon storage at low temperatures.

The following developmental high molecular weight C₄C₁₆ modified HECs provided at least 4 weeks stability to the emulsions at 40° C.: ADPP 6433, ADPP 6435, ADPP 6437, ADPP 6438, 6441, ADPP 6443, and ADPP 6444. (See Table 11.)

TABLE 11
Properties of C₄C₁₆ modified HEC samples
C₄(GE)C₁₆(Br)

| ADPP | MW (Da) | HE-MS | C₄ (wt %) | C₁₆ (wt %) |
|---|---|---|---|---|
| 6433 | 1000K | 3.02 | 1.47 | 0.69 |
| 6435 | 1000K | 3.02 | 2.07 | 0.50 |
| 6437 | 1000K | 3.03 | 2.57 | 0.92 |
| 6438 | 1000K | 3.19 | 3.63 | 0.19 |
| 6441 | 1000K | 3.25 | 4.37 | 0.51 |
| 6443 | 1000K | 2.82 | 4.90 | 0.74 |
| 6444 | 1000K | 3.11 | 4.61 | 1.10 |

Results from Example 1 demonstrated that HECs modified with alkyl chains containing less than sixteen carbons (e.g. C₄, and no modification) showed a marked decrease in their ability to lower interfacial tension and reduce droplet size. Chains shorter than C₁₆ were not sufficiently hydrophobic to provide a strong anchor to an oil droplet. Low molecular weight C₁₆ modified HECs (˜300-500K) were the most efficient emulsifiers. Although high molecular weight HMHECs were only slightly less effective at droplet size reduction, they appeared to be very effective in steric stabilization. Better emulsion stabilization was obtained with high molecular weight polymers. The higher the molecular weight of the polymer, the higher the amount of adsorption and the adsorbed layer thickness were. The oil droplets were completely covered by the polymer chains and the hydrodynamic thickness of the polymer chain was sufficiently large to prevent close approach of the droplets and bridging flocculation. The repulsion between approaching oil droplets started at much larger distances. Next, the fraction of non-adsorbed high molecular weight polymers in the aqueous phase provided higher viscosity and elasticity to the emulsions as a result mainly from more chain entanglements, which also delayed creaming and phase separation of the emulsion.

### EXAMPLE 8

This Example shows the use of mixed hydrophobe HEC in electrolyte containing oil-in-water emulsion. The composition of the emulsion is given in Table 12.

TABLE 12
Oil-in-water emulsion formulation

| Ingredients | Wt % |
|---|---|
| Water Phase | |
| Distilled water | q.s. to 100.0 |
| Glycerin | 5.0 |
| PEG-40 Stearate | 3.0 |
| Sodium chloride | 2.0 |
| Emulsifier/Stabilizer | 1.0 |
| Oil Phase | |
| Carpylic/Capric Triglyceride | 3.3 |
| Octyldodecanol | 3.3 |
| Dicaprylyl Carbonate | 3.3 |
| Phenoxyethanol + Methylparaben + Ethylparaben + | 0.5 |
| Butylparaben + Isobutylparaben + Propylparaben | |

### Procedure:

Polymer (thickener) was added to water and glycerine mixture, while heating (to 50° C.) and mixing (in a blade mixer, @ 700 rpm), until the polymer dissolves completely to form an aqueous solution.
Electrolytes (Sodium chloride) were added to the polymer solution.
PEG-40 stearate was added to the polymer solution to form the aqueous phase.
The oil phase was mixed in a separate flask.
The oil phase was added to the aqueous phase (@ 700 rpm), while cooling to ambient temperature.
The mixture was stirred and homogenized (in a Braun kitchen mixer for 3 minutes at speed 5).

The sodium chloride containing oil-in-water emulsions with C₄C₁₆ modified HECs (ADPP 6266, 6269 and ADPP 6405) were stable for over 12 weeks at 40° C. (See Table 13.)

The sodium chloride containing oil-in-water emulsions with C₈C₂₂ modified HECs (ADPP 4946) were stable for over 12 weeks at 40° C. (See Table 13.)

High molecular weight C₄C₁₆ modified HEC has been shown to function both as emulsifier and steric stabilizer in sodium chloride containing oil-in-water emulsion because of its high molecular weight and mixed hydrophobes. C₁₆ Modification was needed to ensure adsorption of the hydrophobic chains to the oil droplet surface and heterogeneous C₄C₁₆ substitution for self-association of both adsorbed and non-adsorbed polymer. In case the associations were not strong enough to stabilize the system without electrolytes for long term, addition of sodium chloride improved significantly the stability of the emulsions. As a result of stronger association of the C₄C₁₆ hydrophobes in the presence of sodium chloride, the hydrodynamic thickness of adsorbed polymer at the o/w interface was larger and the aqueous phase viscosity of non-adsorbed polymer increased with more elastic structure to prevent creaming of the emulsion.

TABLE 13
Properties of mixed hydrophobe modified HEC samples
C₄(GE)C₁₆(Br)

| ADPP | MW (Da) | HE-MS | C₄ (wt %) | C₁₆ (wt %) |
|---|---|---|---|---|
| 6266 | 1000K | 3.43 | 2.34 | 0.59 |
| 6269 | 1000K | 3.67 | 3.28 | 0.49 |
| 6405 | 1000K | 4.37 | 3.36 | 0.65 |

C₈(GE)C₂₂(GE)

| ADPP | MW | HE-MS | C₈ (wt %) | C₂₂ (wt %) |
|---|---|---|---|---|
| 4946 | 1000K | 2.59 | 0.55 | 0.31 |

### EXAMPLE 9

This Example shows the use of mixed hydrophobe HEC in electrolyte containing oil-in-water emulsion, in which UV-B filter (Phenylbenzimidazol sulfonic acid+Sodium hydroxide) was applied. This UV-B filter Phenylbenzimidazol sulfonic acid formed water soluble salts with the addition of a base such as triethanolamine and sodium hydroxide. The composition of the emulsion is given in Table 14.

TABLE 14
Oil-in-water emulsion formulation

| Ingredients | Wt % |
|---|---|
| Water Phase | |
| Distilled water | q.s. to 100.0 |
| Glycerin | 5.0 |
| PEG-40 Stearate | 3.0 |
| Phenylbenzimidazol sulfonic acid | 2.0 |
| Sodiumhydroxide solution (10%) | 2.8 |
| Emulsifier/Stabilizer | 1.0 |
| Oil Phase | |
| Carpylic/Capric Triglyceride | 3.3 |
| Octyldodecanol | 3.3 |
| Dicaprylyl Carbonate | 3.3 |
| Phenoxyethanol + Methylparaben + Ethylparaben + | 0.5 |
| Butylparaben + Isobutylparaben + Propylparaben | |

### Procedure:

Polymer (thickener) was added to water and glycerine mixture, while heating (to 50° C.) and mixing (in a Braun blade mixer, @ 700 rpm), until the polymer was dissolved completely to form a solution.
Electrolytes (Phenylbenzimidazol sulfonic acid+Sodium hydroxide) were added to polymer solution.
PEG-40 stearate was added to the solution.
The oil phase was mixed in a separate flask.
The oil phase then was added to the water phase while mixing in the Braun mixer (@ 700 rpm), while cooling to ambient temperature.
The mixture was stirred and homogenized (in a Braun kitchen mixer for 3 minutes at speed 5) in order to form an emulsion.

The UV-B filter containing oil-in-water emulsion with C₄C₁₆ modified HEC (ADPP 6405: MW 1000 KDa, HE-MS 4.37, C₄ 3.36 wt %, C₁₆ 0.65 wt %)) was stable for over 12 weeks at 40° C.

### EXAMPLE 10

This Example shows the use of C₄C₁₆ HEC in 20 wt % polar oil-in-water emulsion. The composition of the emulsion is given in Table 15.

TABLE 15
Oil-in-water emulsion (surfactant free) with high polar oil loading

| Ingredients | Wt % |
|---|---|
| Distilled water | q.s. to 100.0 |
| Polymeric emulsifier/stabilizer | 1.00 |
| Caprylic/capric triglyceride | 6.6 |
| Octyldodecanol | 6.6 |
| DicaprylylCarbonate | 6.6 |
| Germaben II (preservative) | 0.20 |

### Procedure:

Prepared a stock solution of polymeric emulsifier/stabilizer. Mix with Heidolph mixer at 800 rpm.
Added oil and Germaben II to the aqueous phase. Mix for 15 minutes. (Heidolph mixer).
Mixed the formulation with Braun kitchen mixer for 3 minutes at speed 5 to form the emulsion.

It was found that 1.0 wt % ADPP 6922 (MW 1000 KDa, HE-MS 4.42, C₄ 3.42 wt %, C₁₆ 0.53 wt %) emulsion was at least stable for 4 weeks at 40° C.

### EXAMPLE 11

This Example shows the use of C₄C₁₆HEC in oil-in-water emulsion using large quantity of ethanol. The composition of the emulsion is given in Table 16.

TABLE 16
Oil-in-water emulsion (surfactant free) with ethanol

| Ingredients | Wt % |
|---|---|
| Distilled water | q.s. to 100.0 |
| Polymeric emulsifier/stabilizer | 1.00 |
| Carnation oil (mineral oil) | 10.00 |
| Ethanol | 50.00 |
| Germaben II (preservative) | 0.20 |

### Procedure:

Prepared stock solution of polymeric emulsifier/stabilizer. Mix with Heidolph mixer at 800 rpm.
Added ethanol to aqueous polymer solution.
Added mineral oil and Germaben II to the aqueous phase. Mixed for 15 minutes. (Heidolph mixer).
Mixed the formulation with Braun kitchen mixer for 3 minutes at speed 5 to formulate emulsion.

It was found that 1.0 wt % ADPP 6922 (MW 1000 KDa, HE-MS 4.42, C₄ 3.42 wt %, C₁₆ 0.53 wt %) emulsion with 50 wt % ethanol was at least stable for 4 weeks at 40° C. The viscosity of the emulsion was approximately 7000 mPa·s (Brookfield, spindle 4, 30 rpm). The texture of the emulsion was very smooth, not gelly and glossy.

### EXAMPLE 12

This Example shows the use of C₄C₁₆HEC in low pH oil-in-water emulsions using glycolic- and lactic acid. The composition of the emulsion is given in Table 17. The pH of emulsions is 4.0.

TABLE 17
Composition oil-in-water emulsion

| Ingredients | Wt % |
|---|---|
| Distilled water | q.s. to 100.0 |
| Polymeric emulsifier/stabilizer | 1.00 |
| Carnation oil (mineral oil) | 10.00 |
| Germaben II (preservative) | 0.20 |
| Glycolic acid/lactic acid | 5.00 |
| NaOH (18%) | To pH 3.8-4.0 |

### Procedure:

Prepared a stock solution of polymeric emulsifier/stabilizer
Added mineral oil and Germaben II to the aqueous phase
Mixed the formulation with a Braun kitchen mixer for 3 minutes at speed 5 to form the emulsion.
Added glycolic acid or lactic acid and mixed for 1 minute
Adjusted the pH with sodium hydroxide solution to 3.8-4.0

It was found that 1.0 wt % ADPP 6922 (MW 1000 KDa, HE-MS 4.42, C₄ 3.42 wt %, C₁₆ 0.53 wt %) emulsions with pH of 4.0 were at least stable for 4 weeks at 40° C.

### EXAMPLE 13

This Example shows the use of C₄C₁₆ HEC in aqueous surfactant based formulations such as shampoos, body washes and shower gels. Several commercial and developmental hydrophobically modified (HM) hydroxyethylcelluloses were evaluated as thickener in an aqueous surfactant formulation. The composition is given in Table 18.

TABLE 18
Composition of aqueous surfactant formulation with pH 5.5-6.0

| Ingredients | Wt % |
|---|---|
| Water | q.s. to 100.00 |
| Sodium laureth sulfate | 7.00 |
| Decyl glucoside | 2.65 |
| Cocamidopropylbetaine | 3.10 |
| Thickener | -.- |
| Germaben II | 0.20 |
| Citric acid | To pH 5.5-6.0 |

Procedure:
Prepared a solution of thickener.
Added sodium laureth sulfate and mixed homogeneously.
Added Decyl glucoside and mixed homogeneosly.
Added cocamidopropylbetaine and mixed homogeneously.
Adjust pH with citric acid to 5.5-6.0
Added Germaben II.

It was found that ADPP 6405 (MW 1000 KDa, HE-MS 4.37, C₄ 3.36 wt %, C₁₆ 0.65 wt %) was compatible in this surfactant solution containing a relatively high level of cocamidopropylbetaine and it gave high viscosities, at which the solutions were slightly hazy. The thickening efficiency of ADPP 6405 was significantly higher compared to Polysurf® 67 cetyl HEC and Natrosol® Plus 330CS HEC.

The high thickening efficiency was greatly affected by its high molecular weight and in less extent to the C₁₆ modification, which led to more inter- and intra polymer association. The medium C₁₆ percentage also provided excellent compatibility of this polymer with surfactants as a result of the hydrophobe interaction of the C₁₆ hydrophobes on the HMHEC with surfactant micelles. Short hydrophobe C₄ chains were not solubilized in the surfactant micelles, but seemed to have a positive contribution to the thickening efficiency. It was assumed that the fraction of polymer hydrophobe associations was not very high in surfactant solution and also not in water.

| HMHEC | MW (KDa) | C₄(wt %) | C₁₆(wt %) | HE-MS |
|---|---|---|---|---|
| ADPP 6405 | 1000 | 3.36 | 0.65 | 4.37 |
| Polysurf ® 67 CS | 600 | — | 0.50 | 2.75 |
| Natrosol ® Plus 330 CS | 300 | — | 0.7 | 3 |
| ADPP 6250 | 300 | 3.46 | 0.69 | 3.78 |
| ADPP 6299 | 500 | 3.23 | 0.63 | 3.49 |
| ADPP 6437 | 1000 | 2.57 | 0.92 | 3.03 |

### EXAMPLE 14

This Example shows the use of ADPP 6926 (MW 1000 KDa, HE-MS 4.20, C₄ 3.42 wt %, C₁₆ 0.71 wt %) in liquid soap with hydrophilic emollients (moisturizing ingredients) such as glycerin an propyleneglycol. The composition of the liquid soap is given in Table 19.

TABLE 19
Composition of liquid soap (pH 6.0)

| Ingredients | | Wt % |
|---|---|---|
| Water | Water | 75.88 |
| Sodium C14-C16 olefin sulfonate | Rhodacal A246L (40%) | 7.50 |
| Sodium Lauroyl sarcosniate | Crodasinic LS30 (30%) | 6.66 |
| Cocamidopropylbetaine | Tegobetaine L7 | 6.66 |
| C₄C₁₆ HEC | ADPP 6926 | 0.80 |
| Glycol monostearate | Estol 3740 | 1.00 |
| Propyleneglycol | Propyleneglycol | 0.50 |
| Glycerin | Glycerin | 0.50 |
| Tetrasodium EDTA | EDTA B Pulver | 0.30 |
| Stearalkonium chloride | Ammonyx 4002 | 0.10 |
| Methyl paraben | Nipagen M | 0.10 |

It was found that the liquid soap formulation with ADPP 6926 was at least stable for 4 weeks at room temperature and 40° C. The viscosity was approximately 3200 mPa·s (Brookfield, spindle 4, 30 rpm).

While this invention has been described with respect to specific embodiments, it should be understood that these embodiments are not intended to be limiting and that many variations and modifications are possible without departing from the scope and spirit of this invention.

What is claimed:

1. An emulsion comprising an oil phase, a water phase, and a mixed hydrophobe, water-soluble, hydrophobically modified polysaccharide composition comprising a water-soluble polysaccharide backbone having at least one C₃-C₈ short chain hydrophobic group and at least one C₉-C₂₄ long chain hydrophobic group attached thereon.

2. The emulsion of claim 1, wherein the backbone of the hydrophobically modified polysaccharide composition is a cellulose ether.

3. The emulsion of claim 2, wherein the cellulose ether is selected from the group consisting of hydroxyethycellulose (HEC), hydroxypropylcellulose (HPC), methylcellulose (MC), hydroxypropylmethylcellulose (HPMC), ethylhydroxyethylcellulose (EHEC), and methylhydroxyethylcellulose (MHEC).

4. The emulsion of claim 1, wherein said the polysaccharide is non-ionic.

5. The emulsion of claim 1, wherein said the polysaccharide can also be modified with anionic, cationic or amphoteric groups.

6. The emulsion of claim 1, wherein the at least one C₃-C₈ short chain hydrophobic group further comprises a C₄ short chain hydrophobic group.

7. The emulsion of claim 1, wherein the at least one C₃-C₈ short chain hydrophobic group further comprises a C₈.

8. The emulsion of claim 1, wherein the at least one C₉-C₂₄ long chain hydrophobic group comprises a C₁₆.

9. The emulsion of claim 1, wherein the at least one C₉-C₂₄ long chain hydrophobic group comprises a C₂₂.

10. A personal care composition comprising (a) a mixed hydrophobe, water-soluble, hydrophobically modified polysaccharide composition comprising a water-soluble polysaccharide backbone having at least one C₃-C₅ short chain hydrophobic group and at least one C₉-C₂₄ long chain hydrophobic group attached thereon and (b) at least one active personal care ingredient.

11. The personal care composition of claim 10, wherein the composition further comprises from about 0.1% to about 99% by weight of the personal care composition of a compatible solvent or solvent mixture.

12. The personal care composition of claim 11, wherein the solvent is selected from the group consisting of water, lower alkanols, polyhydric alcohols having from 3 to 6 carbon atoms and from 2 to 6 hydroxyl groups, and mixtures thereof.

13. The personal care composition of claim 10, wherein the solvent is selected from the group consisting of water, propylene glycol, glycerine, sorbitol, ethanol, and mixtures thereof.

14. A mixed hydrophobe, water-soluble, hydrophobically modified polysaccharide composition comprising a non-ionic water-soluble polysaccharide backbone having at least one C₃-C₅ short chain hydrophobic group and at least one C₉-C₂₄ long chain hydrophobic group attached thereon.

15. The mixed hydrophobe, water-soluble, hydrophobically modified polysaccharide composition of claim 14, wherein the backbone of the hydrophobically modified polysaccharide composition is a cellulose ether.

16. The mixed hydrophobe, water-soluble, hydrophobically modified polysaccharide composition of claim 15, wherein the cellulose ether is selected from the group consisting of hydroxyethycellulose (HEC), hydroxypropylcellulose (HPC), methylcellulose (MC), hydroxypropylmethylcellulose (HPMC), ethylhydroxyethylcellulose (EHEC), and methylhydroxyethylcellulose (MHEC).

17. The mixed hydrophobe, water-soluble, hydrophobically modified polysaccharide composition of claim 15, wherein said the polysaccharide can also be additionally modified with anionic, cationic or amphoteric groups.

18. The mixed hydrophobe, water-soluble, hydrophobically modified polysaccharide composition of claim 15, wherein the at least one C₃-C₅ short chain hydrophobic group further comprises a C₄ short chain hydrophobic group.

19. The mixed hydrophobe, water-soluble, hydrophobically modified polysaccharide composition of claim 15, wherein the at least one C₉-C₂₄ long chain hydrophobic group comprises a C₁₆.
